(19) United States
(12) Patent Application Publication
(10) Pub. No.: US 2020/0124614 A1
(43) Pub. Date: Apr. 23, 2020

(54) Compositions and Methods for Assessing Acute Kidney Injury (AKI) and Mortality Risk

(71) Applicant: YALE UNIVERSITY, New Haven, CT (US)

(72) Inventors: Chirag PARIKH, Lutherville, MD (US); Steven COCA, Norwalk, CT (US)

(21) Appl. No.: 16/591,871

(22) Filed: Oct. 3, 2019

Related U.S. Application Data
(60) Provisional application No. 62/740,747, filed on Oct. 3, 2018.

(51) Int. Cl. G01N 33/68; G16H 50/30; G01N 33/487

(52) U.S. Cl. CPC G01N 33/6827; G16H 50/30; G01N 2333/70578; G01N 2800/347; G01N 33/487

(57) ABSTRACT

The present invention includes methods for treating, preventing, or assessing the levels of risk for acute kidney injury (AKI) in a subject via measuring concentrations of tumor necrosis factor receptor-1 (TNFR1), tumor necrosis factor receptor-2 (TNFR2), and kidney injury molecule-1 (KIM-1).

### CROSS-REFERENCE TO RELATED APPLICATION

The present application claims priority under 35 U.S.C. § 119(e) to U.S. Provisional Patent Application No. 62/740,747 filed Oct. 3, 2018, which is hereby incorporated by reference in its entirety herein.

### STATEMENT REGARDING FEDERALLY SPONSORED RESEARCH OR DEVELOPMENT

This invention was made with government support under HL085757 awarded by the National Institutes of Health. The government has certain rights in the invention.

### BACKGROUND OF THE INVENTION

Acute kidney injury (AKI) is a sudden episode of kidney failure or kidney damage that can happen over the period of a few hours or a few days. AKI causes a build-up of waste products in blood and makes it hard for the kidneys to keep the right balance of fluid in the body. AKI can also affect other organs such as the brain, heart, and lungs. Generally it occurs due to damage to the kidney tissue caused by decreased kidney blood flow (kidney ischemia) from any cause (e.g., low blood pressure), exposure to substances harmful to the kidney, an inflammatory process in the kidney, or an obstruction of the urinary tract that impedes the flow of urine.

AKI is currently diagnosed on the basis of characteristic laboratory findings, such as elevated blood urea nitrogen and creatinine, or inability of the kidneys to produce sufficient amounts of urine. AKI may lead to a number of complications, including metabolic acidosis, high potassium levels, uremia, changes in body fluid balance, and effects on other organ systems, including death.

AKI complicates recovery from cardiac surgery in up to 30% of patients. AKI injures and impairs the function of the brain, lungs, and gut, and places patients at a 5-fold increased risk of death during hospitalization. AKI requiring renal replacement therapy occurs in 2-5% of patients following cardiac surgery and is associated with 50% mortality. For those who recover from renal replacement therapy or even mild AKI, progression to chronic kidney disease in the ensuing months and years is more likely than for those who do not develop AKI. More efficient and time-sensitive methods to diagnose AKI are imperative to reduce this negative outcome. AKI also has significant independent associations with length of stay, costs of hospitalization, and mortality, and the duration and severity of AKI predict higher risk of long-term mortality. Current measures for predicting AKI are unsatisfactory.

A need exists for compositions and methods for diagnosing AKI and predicting the risk of negative outcomes following cardiac surgery. The present invention satisfies this need.

### BRIEF SUMMARY OF THE INVENTION

As described herein, the present invention relates to compositions and methods for assessing acute kidney injury (AKI) and mortality risk.

In one aspect, the invention includes a method of delaying or preventing the development of acute kidney injury (AKI) in a subject. The method comprises measuring concentrations of at least two biomarkers selected from the group consisting of tumor necrosis factor receptor-1 (TNFR1), tumor necrosis factor receptor-2 (TNFR2), and kidney injury molecule-1 (KIM-1) from a sample from the subject The subject's biomarker concentrations are compared with those from a reference sample. When the subject's biomarker concentrations are higher than those from the reference sample, the subject is determined to be at higher risk than average for AKI. In certain embodiments, a therapeutic treatment is administered to the subject, thereby delaying or preventing the development of AKI.

In another aspect, the invention includes a method of treating AKI in a subject. The method comprises measuring concentrations of at least two biomarkers selected from the group consisting of TNFR1, TNFR2, and KIM-1 from a sample from the subject. The subject's biomarker concentrations are compared with those from a reference sample. When the subject's biomarker concentrations are higher than those from the reference sample, the subject is determined to be in need of treatment for AKI. In certain embodiments, a therapeutic treatment for AKI is administered to the subject.

In yet another aspect, the invention includes a method of treating a subject post-cardiac surgery. The method comprises measuring pre-cardiac surgery concentrations of at least two biomarkers selected from the group consisting of TNFR1, TNFR2, and KIM-1 from a sample from the subject, and measuring post-cardiac surgery concentrations of at least two biomarkers selected from the group consisting of TNFR1, TNFR2, and KIM-1 from a sample from the subject. The subject's pre-cardiac surgery biomarker concentrations are compared with the subject's post-cardiac surgery biomarker concentrations. When the subject's pre-cardiac surgery concentrations are higher than the subject's post-cardiac surgery concentrations, the subject is determined to be at higher risk than average for mortality. In certain embodiments, the subject is administered a therapeutic treatment.

In still another aspect, the invention includes a kit useful for assessing the level of risk for AKI in a subject. The kit comprises a bioassay capable of detecting at least one of TNFR1, TNFR2, and KIM-1, and instructional material for use thereof.

In various embodiments of the above aspects or any other aspect of the invention delineated herein, the subject has undergone cardiac surgery. In certain embodiments, the subject's biomarker concentrations are measured pre-cardiac surgery.

In certain embodiments, the therapeutic treatment is selected from the group consisting of medication, fluids, contra-statins, blood transfusions, and renal replacement therapy.

In certain embodiments, the biomarker concentrations are measured via a bioassay.

In certain embodiments, the subject is a human.

In certain embodiments, the sample is a plasma sample.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 is a plot showing TNFR-1 concentrations over time.

FIG. 2 is a plot showing TNFR-2 concentrations over time.

FIG. 3 is a plot showing KIM-1 concentrations over time.

FIG. 4 is a table showing the adjusted odds ratios per log increases in biomarker concentrations. *Adjusted for recipient age (per year), recipient gender, white race (Yes/No), CPB time >120 Mins (Yes/No), non-elective surgery, surgery type, pre-op eGFR, diabetes, hypertension, CHF, history of MI, pre-op urine albumin to creatinine ratio, and clinical site. †Adjusted for above variables plus pre-operative biomarker concentration, and change in serum creatinine from baseline to peak post-operative. NA—not applicable.

FIG. 5 is a table showing patient characteristics for studies described elsewhere herein.

FIG. 6 is a table showing a summary of acute kidney injury (AKI) & mortality results. *Adjusted for recipient Age (per year), recipient gender, white race (Yes/No), CPB time >120 Mins (Yes/No), non-elective surgery, surgery type, pre-op eGFR, diabetes, hypertension, CHF, history of MI, pre-op urine albumin to creatinine ratio, and clinical site. †Adjusted for above variables plus pre-operative biomarker concentration, and change in serum creatinine from baseline Median (P25, P75). Number of observations and Wilcoxon P-values for all variables are presented.

FIG. 7 is a table showing biomarker distribution by AKI. For each patient a pre-op, day 1 0-6 hours and a second post-op sample (96% day 2, 3% day 3, 1% day 4) were selected. The peak value is calculated as the highest value from the 2 post-op samples. **P value columns with light grey background means the value is less than 0.05. “Ratio of Day1 Pos-Ope/Pre-Ope” is the ratio of measured values at day 1 post-operation versus the value measure at pre-operation. “Ratio of Peak Pos-Ope/Pre-ope” is the ratio between peak value post-operation and pre-operation. AKI defined at AKIN Stage 1 or higher (>50% or >0.3 mg/dL or dialysis) to peak post-operative.

FIGS. 8A-8C are a series of tables showing odds ratios of log transformed and tertiled values for each time point of each biomarker (AKI (Yes/No) as outcome). Odds ratios of AKI (Yes/No) for log-transformed continuous biomarkers and tertiled biomarkers are presented in this table. ‘1 vs 0’ means odds ratio of AKI comparing tertiled biomarkers middle group with lower group. The biomarkers are measured at 3 time points: pre-operation, Day 1 (1-6 hours) after operation, Day2 (or day3, day4) after operation. Peak means higher values out of day1 and day2 (or day3, day4). Peak values distribution varies for different biomarkers. For example, peak values distribution of YKL40 is: 37 (3%) at day 1; 1014 (92%) at day 2; 40 (4%) at day 3; 11 (1%) at day 4. # of Event without covariates is the number of AKI in unadjusted model. The total number of patients is 1444. # of Event with covariates is the number of AKI in adjusted 1, 2, 3 models (N=1407, 37 missing values), because some covariates in the model have missing values (Myocardial Infarction has 19 missing values; Pre-op urine albumin to creatinine ratio has 16 missing values, CPB time has 3 missing values). Unadjusted OR only includes biomarkers as independent variable. Adjusted 1 is adjusted for clinical covariates including: recipient Age (per year), recipient gender, white race (Yes/No), CPB time >120 Mins (Yes/No), non-elective surgery, surgery type, pre-op eGFR, diabetes, hypertension, Congestive heart failure (chf), Myocardial Infarction (mi), Pre-op urine albumin to creatinine ratio, site. The covariate CPB time >120 isn't included in pre-operation time point. Adjusted 2 is adjusted for adjusted1+corresponding pre-operation biomarker (Only for day1 0-6 hours and Peak groups). Adjusted 3 is adjusted for adjusted2+change in (day1 or peak) serum creatinine from pre-op ((Only for day1 0-6 hours and Peak groups). Adjusted 4 is adjusted for adjusted3+log-transformed IL10 (Except IL10). Adjusted 5 is adjusted for adjusted3+log-transformed IL6 (Except IL6). Adjusted6 is adjusted for adjusted3+Pre-op Steroid use (Yes/No). P-value columns with grey background indicate the value is less than 0.05. Median (P25, P75), number of observations and Wilcoxon P-value for all variables are presented.

FIG. 9 is a table showing biomarker distribution and 1-year mortality. For each patient pre-op, day 1 0-6 hours and a second post-op sample (96% day 2, 3% day 3, 1% day 4) were selected. The peak value is calculated as the highest value from the 2 post-op samples. “Ratio of Day1 Pos-Ope/Pre-Ope” is the ratio of measured values at day 1 post-operation versus the value measure at pre-operation. “Ratio of Peak Pos-Ope/Pre-ope” is the ratio between peak value post-operation and pre-operation. P-value columns with light grey background indicate the value is less than 0.05.

FIGS. 10A-10C are a series of tables showing odds ratios of log transformed and tertiled values for each time point of each biomarker (1-year mortality (Yes/No) as outcome). ‘1 vs 0’ means odds ratio of 1-year mortality comparing tertiled biomarkers middle group with lower group. The biomarkers are measured at 3 time points: pre-operation, Day 1 (1-6 hours) after operation, Day2 (or day3, day4) after operation. Peak means higher values out of day1 and day2 (or day3, day4). Peak values distribution varies for different biomarkers. For example, peak values distribution of YKL40 is: 37 (3%) at day 1; 1014 (92%) at day 2; 40 (4%) at day 3; 11 (1%) at day 4. # of Event without covariates is the number of 1-year mortality in unadjusted model. The total number of patients is 1408. # of Event with covariates is the number of 1-year mortality in adjusted 1, 2, 3 models (N=1377, 31 missing values), because some covariates in the model have missing values (Myocardial Infarction has 19 missing values; Pre-op urine albumin to creatinine ratio has 14 missing values, CPB time has 3 missing values). Unadjusted OR only includes biomarkers as independent variable. Adjusted 1 is adjusted for clinical covariates including: recipient Age (per year), recipient gender, white race (Yes/No), CPB time >120 Mins (Yes/No), non-elective surgery, surgery type, pre-op eGFR, diabetes, hypertension, Congestive heart failure (chf), Myocardial Infarction (mi), Pre-op urine albumin to creatinine ratio, site. The covariate CPB time >120 isn't included in pre-operation time point. Adjusted 2 is adjusted for adjusted1+corresponding pre-operation biomarker (Only for day1 0-6 hours and Peak groups). Adjusted 3 is adjusted for adjusted2+change in (day1 or peak) serum creatinine from pre-op ((Only for day1 0-6 hours and Peak groups). Adjusted 4 is adjusted for adjusted3+log-transformed IL10 (Except IL10). Adjusted 5 is adjusted for adjusted3+log-transformed IL6 (Except IL6). Adjusted6 is adjusted for adjusted3+Pre-op Steroid use (Yes/No). P-value columns with grey background means the value is less than 0.05.

FIGS. 11A-11C are a series of tables showing odds ratios of 1-year mortality (Yes/No) for log-transformed continuous biomarkers and tertiled biomarkers interaction with AKI (Yes/No). The biomarkers are measured at 3 time points: pre-operation, Day 1 (1-6 hours) after operation, Day2 (or day3 day4) after operation. Peak means higher values out of day1 and day2 (or day3, day4). Unadjusted OR only includes biomarker values as independent variable. Adjusted 1 is adjusted for clinical covariates including: recipient Age (per year), recipient gender, white race (Yes/No), CPB time >120 Mins (Yes/No), non-elective surgery, surgery type, pre-op eGFR, diabetes, hypertension, Congestive heart failure (chf), Myocardial Infarction (mi), Pre-op urine albumin to creatinine ratio, site, corresponding pre-operation biomarkers and change in serum creatinine of day1 (or peak) from pre-op creatinine. CPB time >120, corresponding pre-operation biomarkers and change in serum creatinine of day1 (or peak) from pre-op creatinine are not included in pre-operation time point. Type 3 p value indicates whether the odds ratio is significantly different between AKI No group and AKI Yes group. If they are significantly different, the type3 p value is less than 0.05 and is indicated by a dark grey background. P values less than 0.05 are marked with a light grey background.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplified materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of an immunogenic composition includes, e.g., subcutaneous (s.c.), intravenous (i.v.), intramuscular (i.m.), or intrasternal injection, or infusion techniques.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides methods for assessing the levels of risk for acute kidney injury (AKI) and/or mortality in a subject via measuring concentrations of tumor necrosis factor receptor-1 (TNFR1), tumor necrosis factor receptor-2 (TNFR2), and kidney injury molecule-1 (KIM-1) in a biological sample from the subject. In certain embodiments, if the subject is found to be at risk of AKI, the subject is provided with appropriate medical counseling and/or therapeutic interventions.

One pathway of inflammation that has emerged as central in DKD involves tumor necrosis factor. Gene expression studies have revealed that CKD risk-associated transcripts showing an inverse correlation with eGFR clustered around TNF-alpha. TNF-alpha directly stimulates podocytes to produce several cytokines, utilizing TNF receptors (TNFRs). TNF cell surface receptors are shed into the extracellular space, including into blood, after cleavage with TNF-alpha cleaving enzyme. In clinical studies, plasma TNFR1 and TNFR2 concentrations have been shown to be associated with the development of incident CKD, progressive CKD, and end stage renal disease (ESRD) in type 1 and type 2 diabetics.

Kidney injury molecule (KIM)-1 is expressed in the apical membrane of proximal tubular cells in response to injury and promotes kidney fibrosis. KIM-1 may enter the circulation because of increased trans-epithelial permeability or loss of epithelial cell polarity with basolateral membrane expression in early injury. In a study of type 1 diabetics, plasma KIM-1 levels predicted the rate of eGFR loss and ESRD risk during a mean of 10 years of follow-up, after adjustment for confounders. Plasma KIM-1 has been associated with incident CKD in type 2 diabetics with preserved GFR and progressive CKD in type 2 diabetics with prevalent diabetic nephropathy.

### Methods

In one aspect, the invention includes a method of assessing the level of risk for developing acute kidney injury (AKI) in a subject. The method comprises measuring the concentrations of at least two biomarkers selected from the group consisting of tumor necrosis factor receptor-1 (TNFR1), tumor necrosis factor receptor-2 (TNFR2), and kidney injury molecule-1 (KIM-1) from a sample from the subject. The subject's biomarker concentrations are compared with those from a reference sample. When the subject's biomarker concentrations are higher than those from the reference sample, the subject is determined to be at high risk for AKI. In certain embodiments, for each log increase in the biomarker the risk of AKI increases by 2-3 fold. This is a continuous risk across the range of values.

In one aspect, the invention includes a method of delaying or preventing the development of acute kidney injury (AKI) in a subject. The method comprises measuring the concentrations of at least two biomarkers selected from the group consisting of tumor necrosis factor receptor-1 (TNFR1), tumor necrosis factor receptor-2 (TNFR2), and kidney injury molecule-1 (KIM-1) from a sample from the subject. The subject's biomarker concentrations are compared with those from a reference sample. When the subject's biomarker concentrations are higher than those from the reference sample, the subject is determined to be at high risk for AKI and is administered a therapeutic treatment, thereby delaying or preventing the development of AKI.

In another aspect, the invention includes a method of treating AKI in a subject. The method comprises measuring the concentrations of at least two biomarkers selected from the group consisting of TNFR1, TNFR2, and KIM-1 from a sample from the subject. The subject's biomarker concentrations are compared with those from a reference sample. When the subject's biomarker concentrations are higher than those from the reference sample, the subject is determined to be in need of treatment for AKI, and a treatment is administered to the subject.

In certain embodiments, the subject has undergone cardiac surgery and the concentrations of TNFR1, TNFR2, and KIM-1 in the subject are measured pre-surgery. In other embodiments, the subject has undergone a surgery other than cardiac surgery. It should be understood by one of ordinary skill in the art that the methods of the invention are to be construed to include any type of surgery wherein the subject would be at risk for developing AKI and/or exacerbating the symptoms of AKI, for example, any abdominal surgery or other high risk surgeries that require general anesthesia. In addition, the methods of the invention are useful for subjects with any condition that can lead to the development or exacerbation of AKI.

In yet another aspect, the invention includes a method of assessing the level of risk for mortality in a subject post-cardiac surgery. The method comprises measuring pre-cardiac surgery concentrations of at least two biomarkers selected from the group consisting of TNFR1, TNFR2, and KIM-1 from a sample from the subject, and then measuring post-cardiac surgery concentrations of at least two biomarkers selected from the group consisting of TNFR1, TNFR2, and KIM-1 from a sample from the subject. The pre-cardiac surgery biomarker concentrations are compared with the post-cardiac biomarker surgery concentrations. When the pre-cardiac surgery concentrations are higher than the post-cardiac surgery concentrations, the subject is determined to be at higher than average risk for mortality due to AKI development. In certain embodiments, for each log increase in the biomarker level in the pre-operative sample the risk of mortality increases by 2 to 2.5 fold. In certain embodiments, for each log increase in the post-operative biomarker level, the risk increases by 3-5 fold.

Another aspect of the invention includes a method of treating a subject post-cardiac surgery. The method comprises measuring pre-cardiac surgery concentrations of at least two biomarkers selected from the group consisting of TNFR1, TNFR2, and KIM-1 from a sample from the subject, and then measuring post-cardiac surgery concentrations of at least two biomarkers selected from the group consisting of TNFR1, TNFR2, and KIM-1 from a sample from the subject. The pre-cardiac biomarker surgery concentrations are compared with the post-cardiac biomarker surgery concentrations. When the pre-cardiac surgery concentrations are higher than the post-cardiac surgery concentrations, the subject is determined to be at higher risk than average for mortality, and a therapeutic treatment is administered to the subject.

In certain embodiments, the methods of the invention further comprise administering a therapeutic treatment to the subject. In some cases, the treatment involves treating an underlying cause of AKI or treating an underlying disorder that contributes to development of AKI. In some instances treatment involves supportive care. Treatment can involve administering to the subject fluids such as intravenous fluids, vasopressors such as norepinephrine, ionotropes such as dobutamine, steroids, cyclophosphamide, or plasma exchange. In certain embodiments, for example in the cases of toxin-induced prerenal AKI, the treatment comprises discontinuing certain agents that exacerbate risk for, or exacerbate severity of, AKI such as ACE inhibitors, ARB antagonists, aminoglycosides, penicillins, NSAIDs, or paracetamol. In certain embodiments, renal replacement therapy, such as with hemodialysis, is instituted. Renal replacement therapy can be applied intermittently (IRRT) or continuously (CRRT). In certain embodiments, treatment is selected from the group consisting of medication, fluids, contra-statins, blood transfusions, and renal replacement therapy. In one non-limiting example, for pre-operative prediction, the surgeons can use techniques such as off-pump cardiac surgery that would reduce the risk of surgery. The administration of treatment can be carried out by standard methods known to those of skill in the art.

In some embodiments, the method further comprises the use of clinical prognostic tools to predict renal decline.

In certain embodiments, the subject is a human. In other embodiments, the sample is a blood sample. In yet other embodiments, the sample is a plasma sample.

### Bioassays

In certain embodiments, the invention includes measuring TNFR1, TNFR2, and KIM-1 via a bioassay. Bioassay is meant to mean any method capable of determining the concentration of a substance. The absolute concentrations can be determined or the relative concentrations can be determined by comparing with that of a standard preparation. Examples of bioassays include but are not limited to immunoassay, ELISA, RIA, ELISPOT, microarray, or biochip. In one embodiment, the bioassay comprises the MesoScale Discovery multiplex assay. Bioassays are performed by routine methods known to those of ordinary skill in the art. In certain embodiments, there could be point of care assays. In other embodiments, the tests can be configured on existing automated analyzers such as Abbott, Roche machines.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### Experimental Examples

The invention is now described with reference to the following Examples. These Examples are provided for the purpose of illustration only, and the invention is not limited to these Examples, but rather encompasses all variations that are evident as a result of the teachings provided herein.

The materials and methods employed in the present invention are now described.

### Participant Characteristics:

TRIBE-AM Cohort:

This was an ancillary observational study from the TRIBE-AKI cardiac surgery cohort, which has been described in detail previously. 1444 adults undergoing cardiac surgery (coronary artery bypass grafting [CABG] or valve surgery) who were at high risk for AKI were prospectively enrolled at six academic medical centers in North America between July 2007 and December 2010. Day 1 was the day of surgery, day 2 corresponded to the first day after surgery. The first postoperative samples were collected soon after admission to the intensive care unit (ICU) within 6 hours after surgery (0-6 hour sample). Subsequently, blood samples were obtained at the time of routine morning blood collection done for clinical care. A total of three time points were measured on all participants; pre-operative, and postoperatively on day 1 (0-6 hours), and peak of first 3 post-operative days. For this ancillary study, biomarkers were measured at the ‘preoperative’ time point—within 24 hours of surgery—and ‘postoperative day 1’ in which the sample was collected within six hours after surgery.

Biomarker Measurement:

Samples underwent a single controlled thaw, were centrifuged at 5000×g for 10 minutes at 4° C., separated into 1 ml aliquots, and immediately stored at −80° C. until biomarkers were measured. Details on the effect of freeze thaw cycles and biomarker measurements, assay performance and stability has been described. The biomarkers TNFR1, TNFR2, and KIM-1 were measured using the Meso Scale Discovery platform (Meso Scale diagnostics, Gaithersburg, Md.), which uses electrochemiluminescence detection combined with patterned arrays.

Outcome Definitions:

There were three primary outcomes for the study. The first was the development of AKI, defined as >0.3 mg/dL increase or 50% increase in serum creatinine from baseline preoperative level to postoperative level at any time point during the hospital stay, which is consistent with the definition of stage 1 AKI based on the Acute Kidney Injury Network (AKIN) classification. All preoperative creatinine values were measured within 2 months before surgery (median:3 days, IQR:0,14). The second outcome was duration of AKI, defined by the number of days participants maintained stage 1 or higher AKI based on AKIN classification of >0.3 mg/dL increase or 50% increase in serum creatinine. AKI duration was dichotomized into <7 days or ≥7 days. The third outcome was one-year all-cause mortality after surgery. Vital status was obtained after discharge through various mechanisms (and cross-referenced when possible). For those living in the United States, phone calls were made to patients' homes, the National Death Index searched, hospital records reviewed, and linkages with Center for Medicare and Medicaid Services (CMS) databases made. For Canadian participants (those enrolled into the TRIBE-AKI study in London, Ontario), phone calls were made and data held at the Institute for Clinical Evaluative Sciences analyzed to acquire vital status. These datasets were linked using unique, encoded identifiers and analyzed at the Institute for Clinical Evaluative Sciences (ICES).

Variable Definitions:

Preoperative characteristics, operative details, and postoperative complications were collected using definitions of the Society of Thoracic Surgeons (wwwdotctsnetdotorg/file/rptDataSpecifications252_1_ForVendorsPGSdotpdf). Preoperative GFR was estimated using the CKD Epidemiology Collaboration equation.

Statistical Analyses:

All analyses were two-tailed and p-values less than 0.05 were considered significant. Descriptive statistics for continuous variables were reported as mean (standard deviation) or median (interquartile range) and for categorical variables as frequencies (%). Participant characteristics were analyzed using Mann-Whitney Wilcoxon tests for continuous variables and chi-squared test for categorical variables. When evaluating the association between angiogenesis biomarkers and outcomes, biomarkers were analyzed both as continuous (loge transformed biomarker levels) and categorical (tertiles) variables. To evaluate the association between each biomarker and all dichotomous outcomes, logistic regression models were used. Biomarker values below the limit of detection were imputed with the limit of detection. Pearson correlations were performed among the three angiogenesis biomarkers at both time points (preoperative and postoperative). Both univariable and multivariable analyses were evaluated. Multivariable analyses were adjusted for the following variables: age in years, gender, white race, CPB time >120 minutes (excluded when assessing associations between preoperative biomarkers and outcomes), non-elective surgery, type of surgery (CABG or valve surgery), preoperative eGFR, diabetes, hypertension, heart failure, myocardial infarction, preoperative urine albumin to creatinine ratio, center, corresponding pre-operative biomarker (adjusted for postoperative time point only), change in serum creatinine from preoperative baseline level to the corresponding biomarker time point measurement (only adjusted for postoperative time point for outcome of mortality and not AKI, as change in serum creatinine is used to define AKI outcomes). Small cell counts are only presented for data collected by TRIBE-AKI and not from ICES data holdings (for the latter, counts of 5 or fewer participants are suppressed to minimize the risk of participant re-identification). Analyses were performed in SAS version 9.3 (SAS Institute, Cary, N.C.) and R 2.15.0 (R Foundation for Statistical Computing, Vienna Austria).

### Example 1

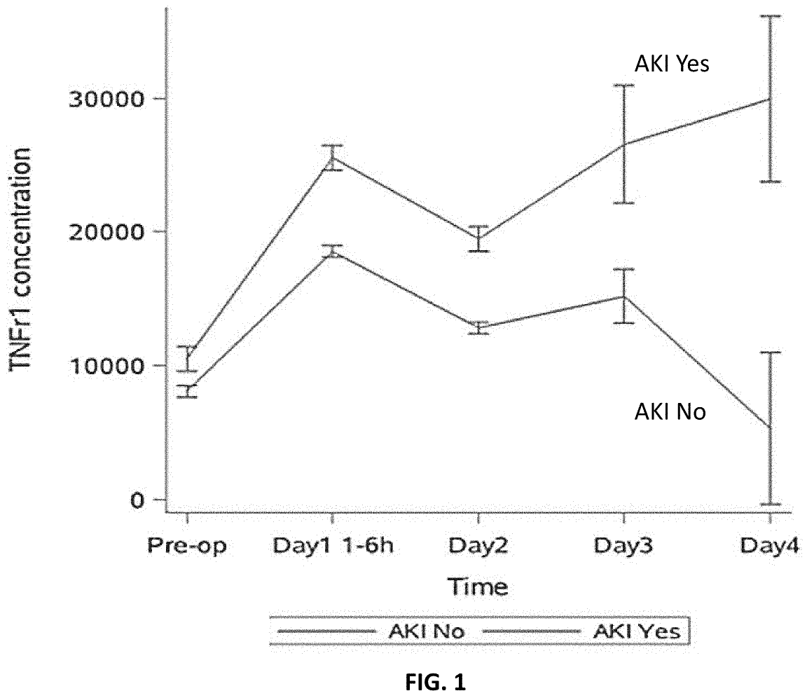

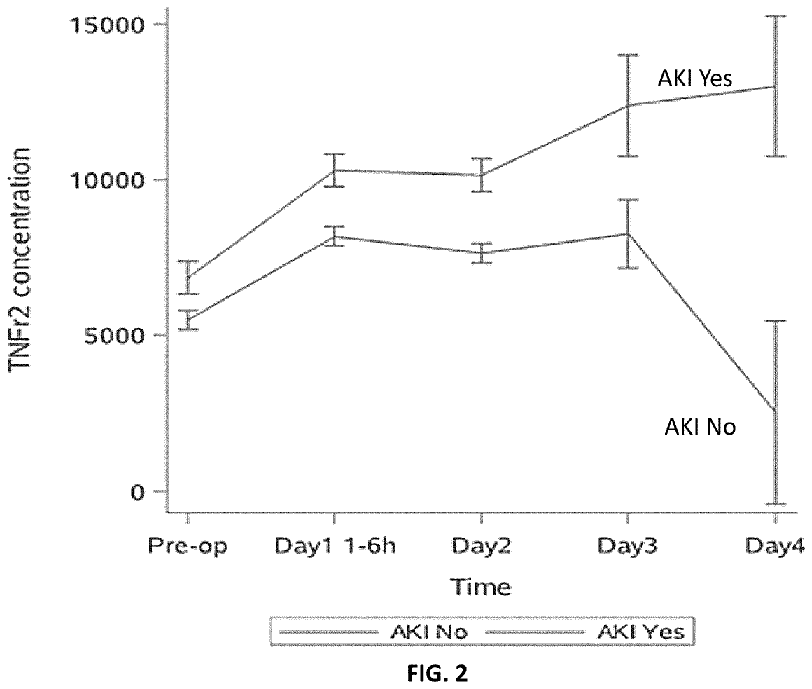

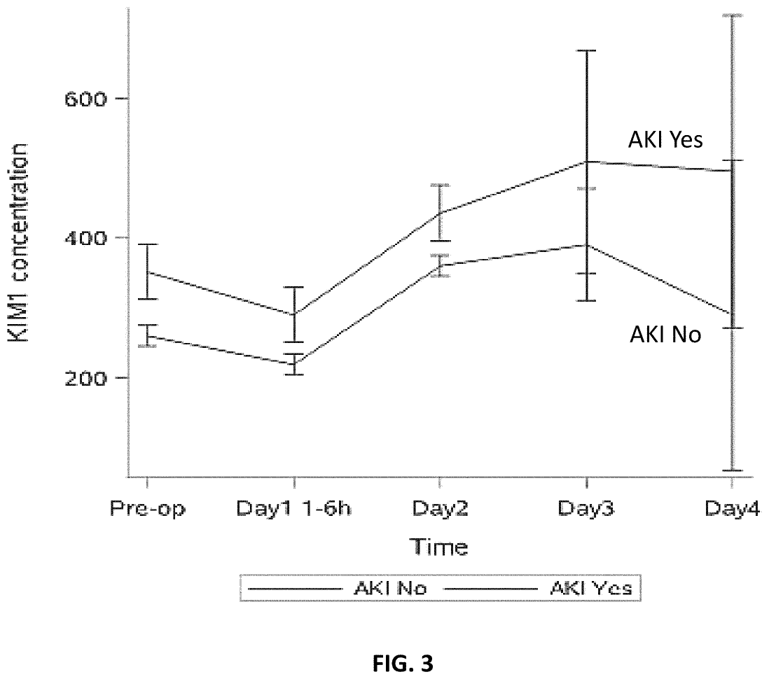

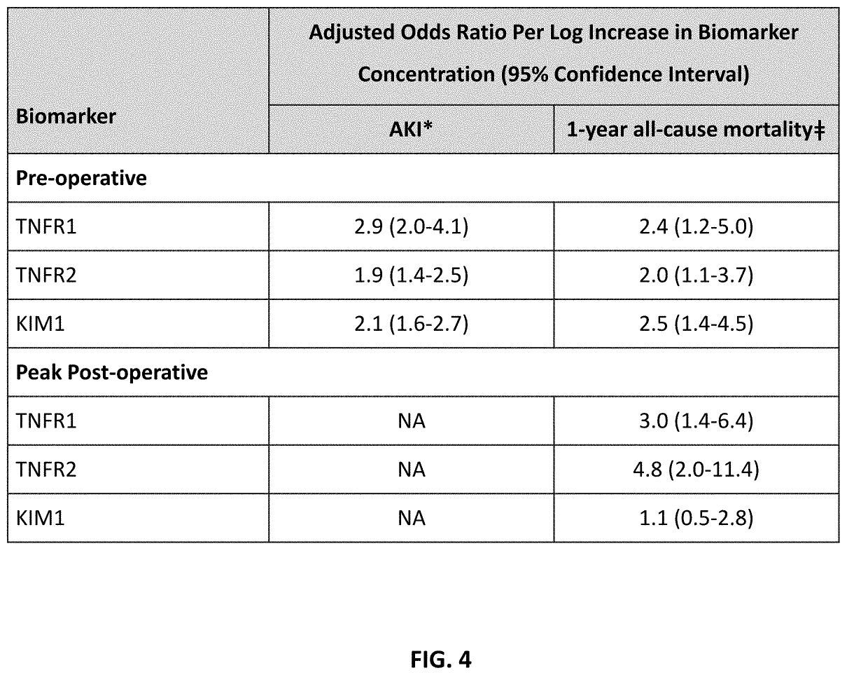

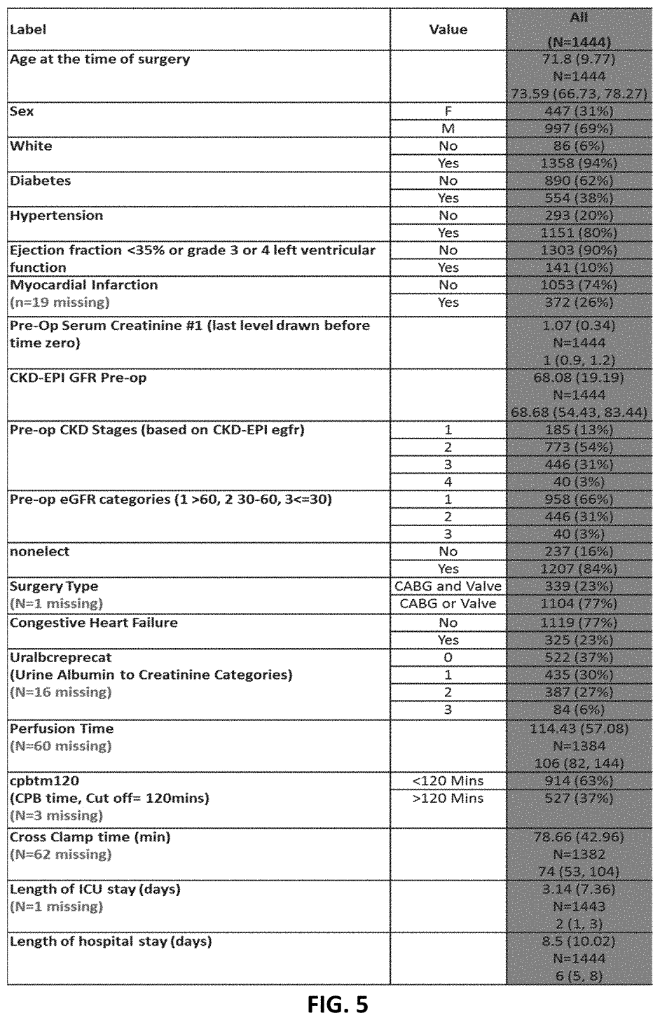

A prospective cohort study of 1,444 high-risk adults undergoing cardiac surgery (CABG, valve, or both) (FIG. 5) was assessed. The associations of pre- and post-operative (peak days 1-3) concentrations of TNFR1, TNFR2, and KIM-1 with post-operative acute kidney injury (AKI) (AKIN stage 1) and 1-year all-cause mortality after cardiac surgery were assessed in the patients. Plasma TNFR1, TNFR2 and KIM-1 were measured via MesoScale Discovery multiplex assay (FIGS. 1-3). Pre-operative concentrations of TNFR1, TNFR2, and KIM-1 were higher in those who developed post-operative AKI (n=492, 34%) and those who died (n=68, 6.2%) by one-year. Each log-increase of pre-operative biomarker was independently associated with a 2-3 fold higher odds of both AKI and one-year mortality (FIG. 4). TNFR1, TNFR2, and KIM-1 concentrations increased by 136, 65, and 36%, respectively, from pre- to post-operatively, and differed minimally by AKI status. After adjustment for pre-operative biomarker value, peak change in serum creatinine and 13 other covariates, peak post-operative levels of TNFR1 and TNFR2, but not KIM-1, were associated with one-year mortality (FIG. 4). The panel of TNFR1, TNFR2, and KIM1 provided strong prognostic information about AKI risk and 1 year mortality when measured preoperatively in patients undergoing cardiac surgery. Moreover, peak post-operative concentrations of TNFR1 and TNFR2 provided additional prognostic information for death.

Experiments described herein assessed the use of plasma TNFR1, TNFR2, and KIM-1 for new outcomes: to predict AKI and mortality in patients undergoing cardiac surgery, including their ability to do so above and beyond clinical variables alone. Plasma biomarkers were measured in stored samples from the TRIBE-AKI cohort and their association with AKI and 1-year mortality after surgery were tested.

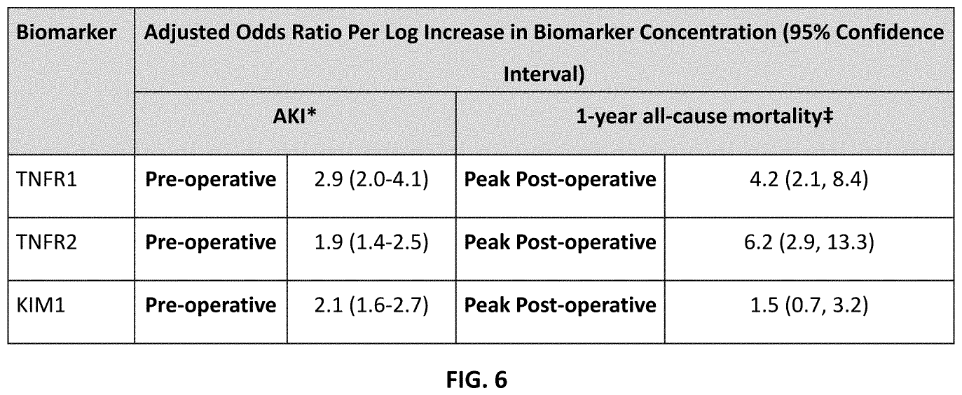

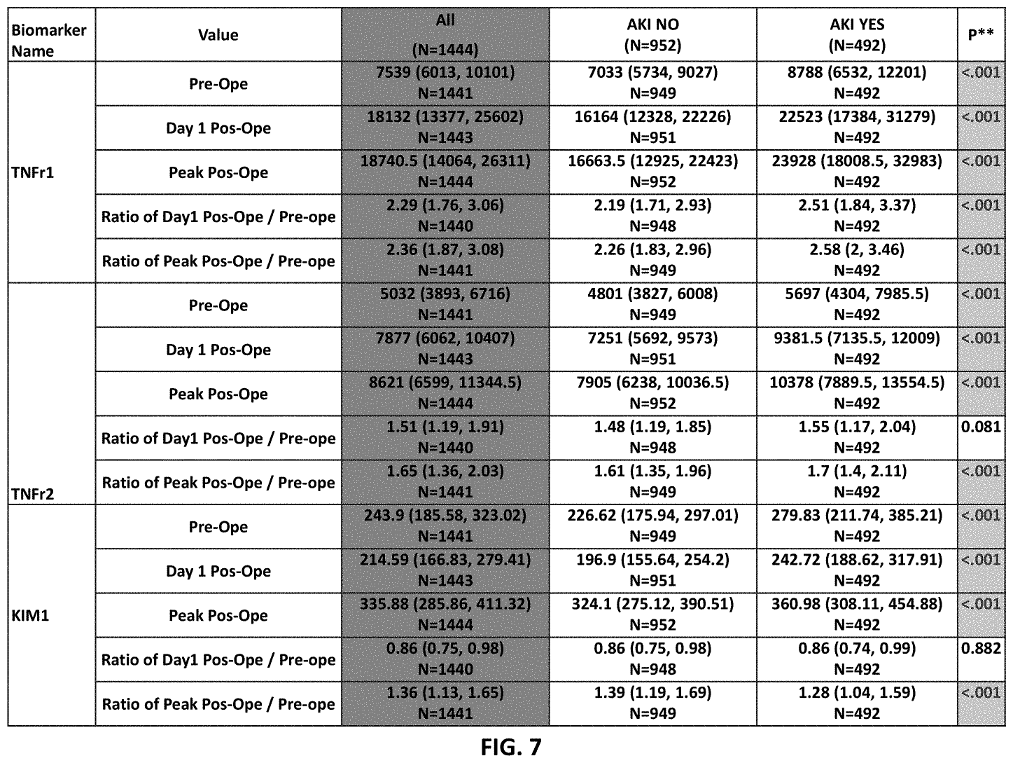

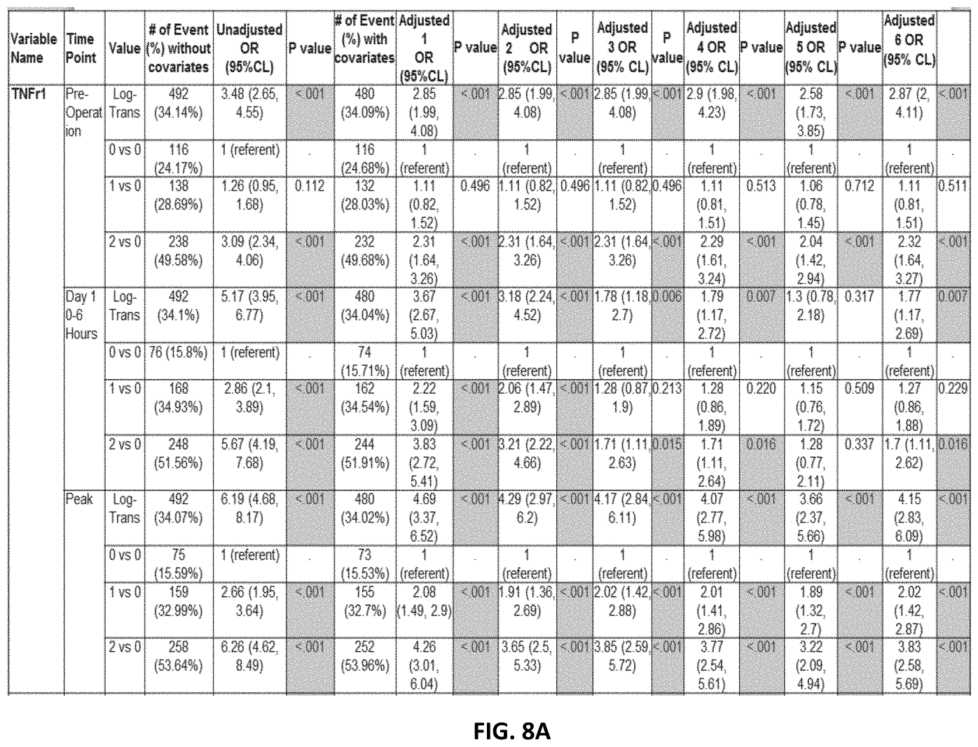

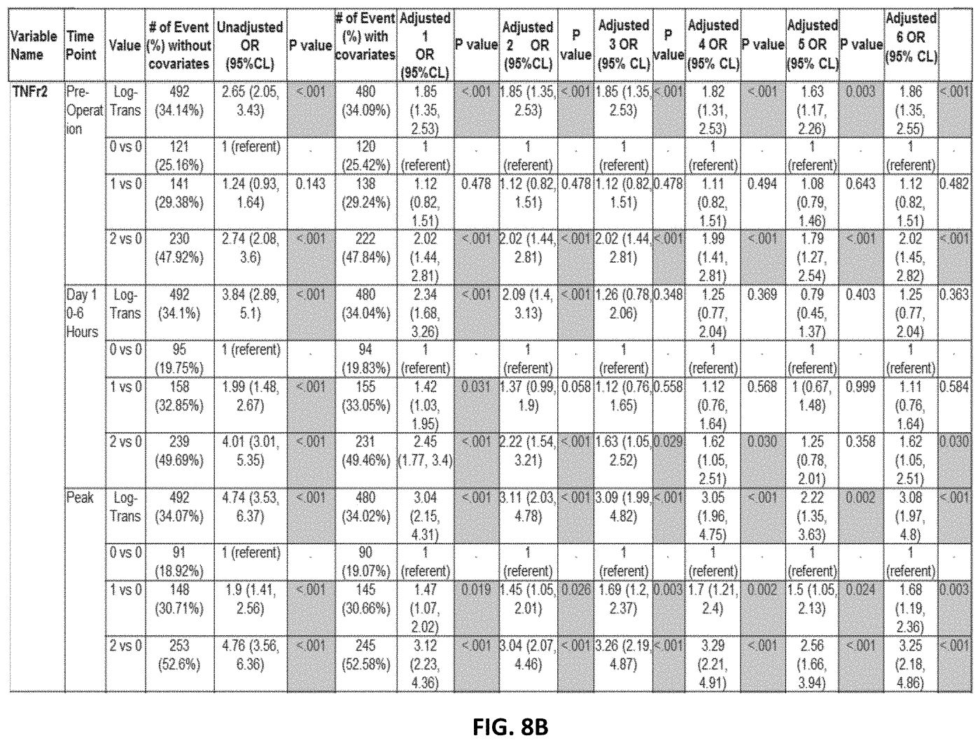

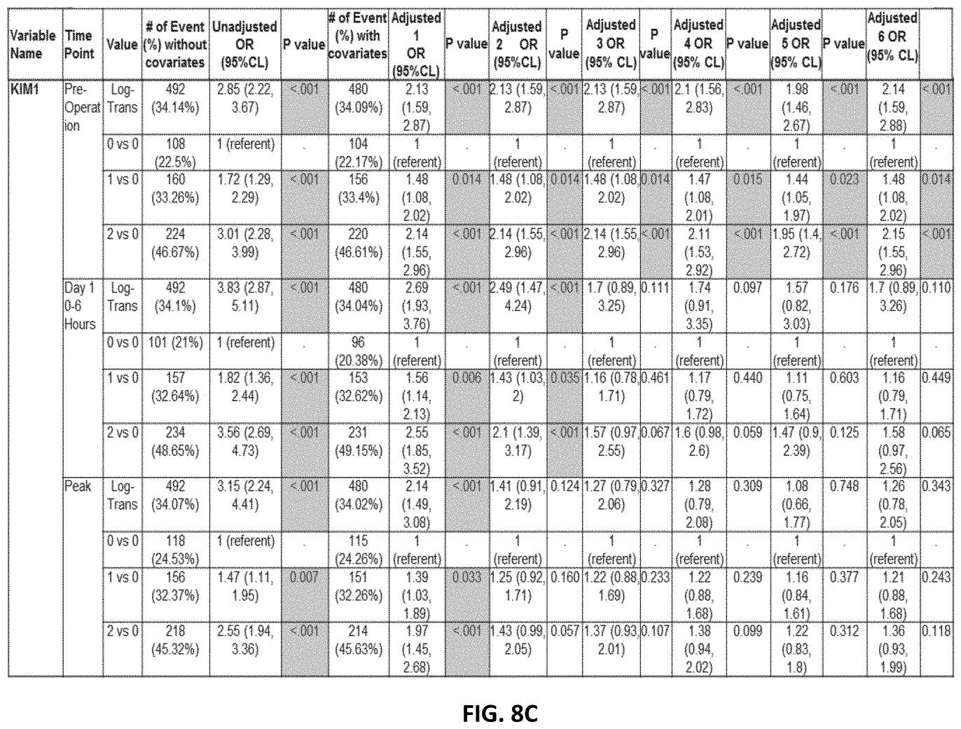

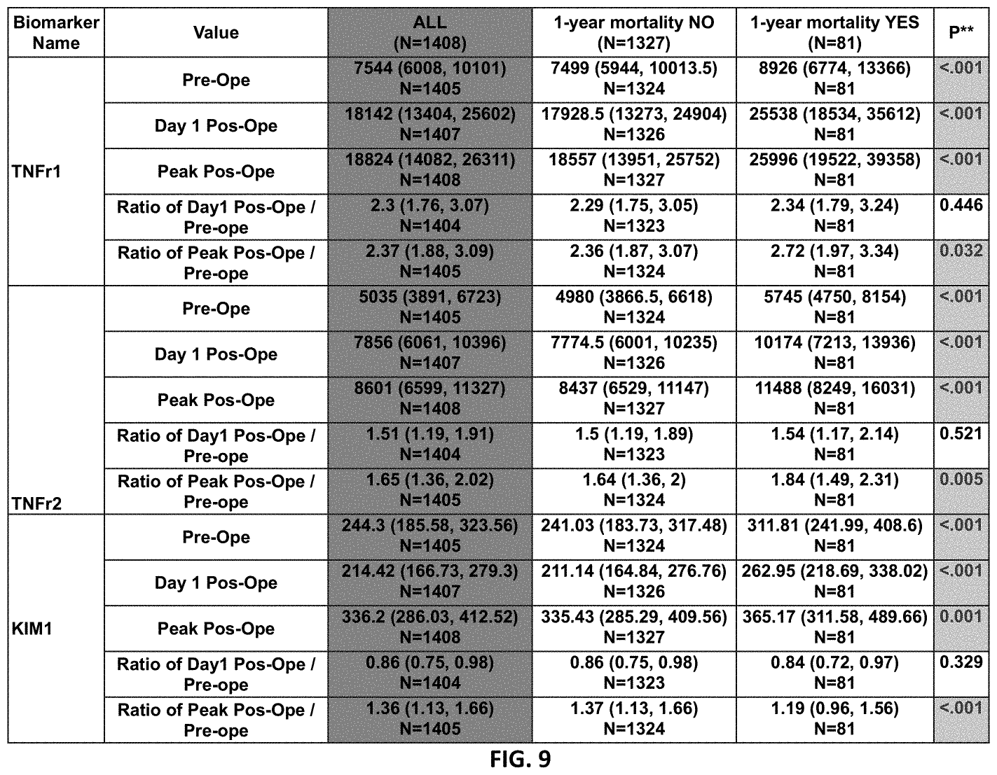

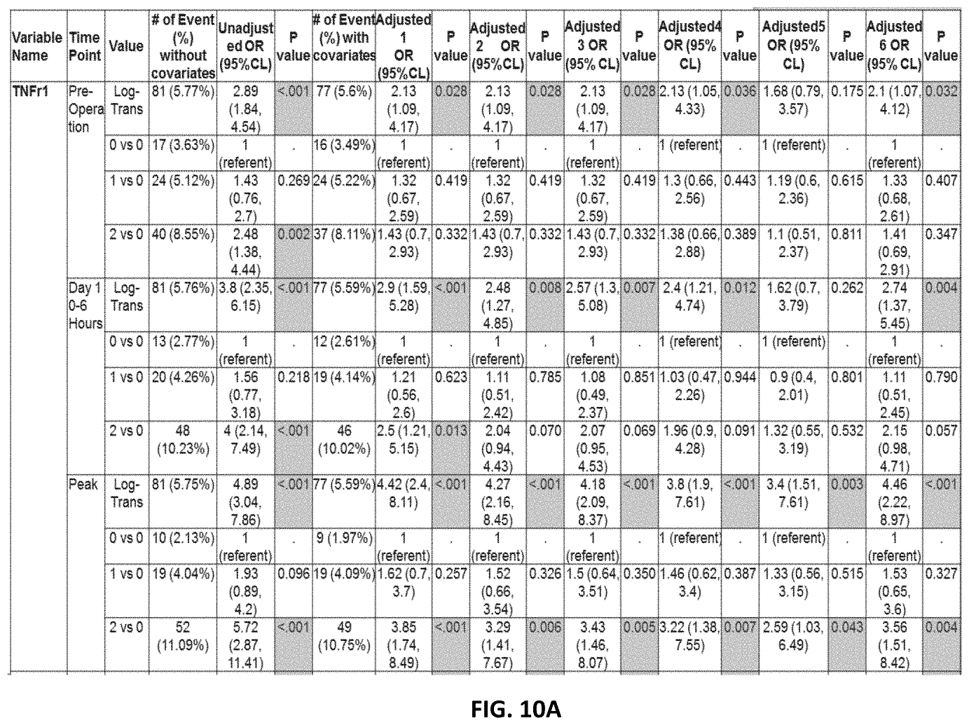

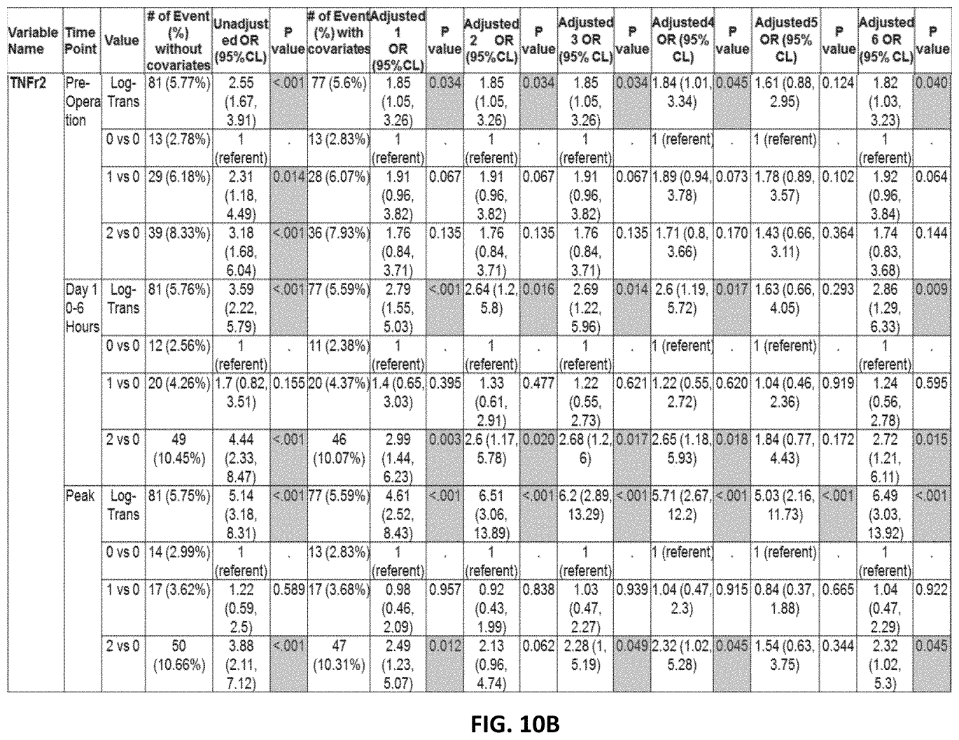

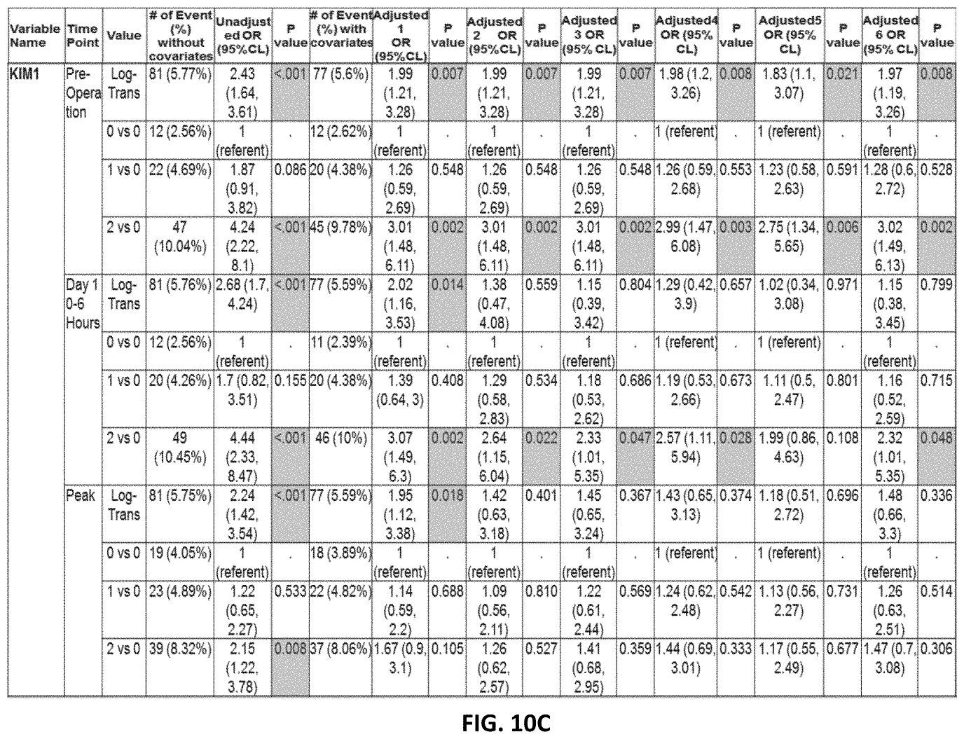

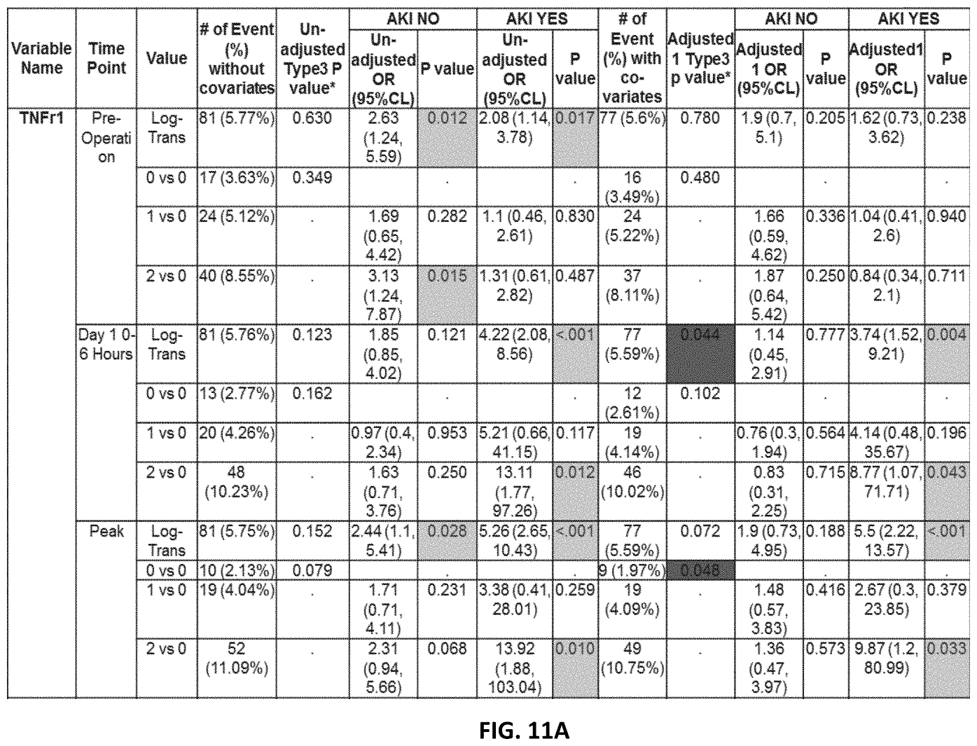

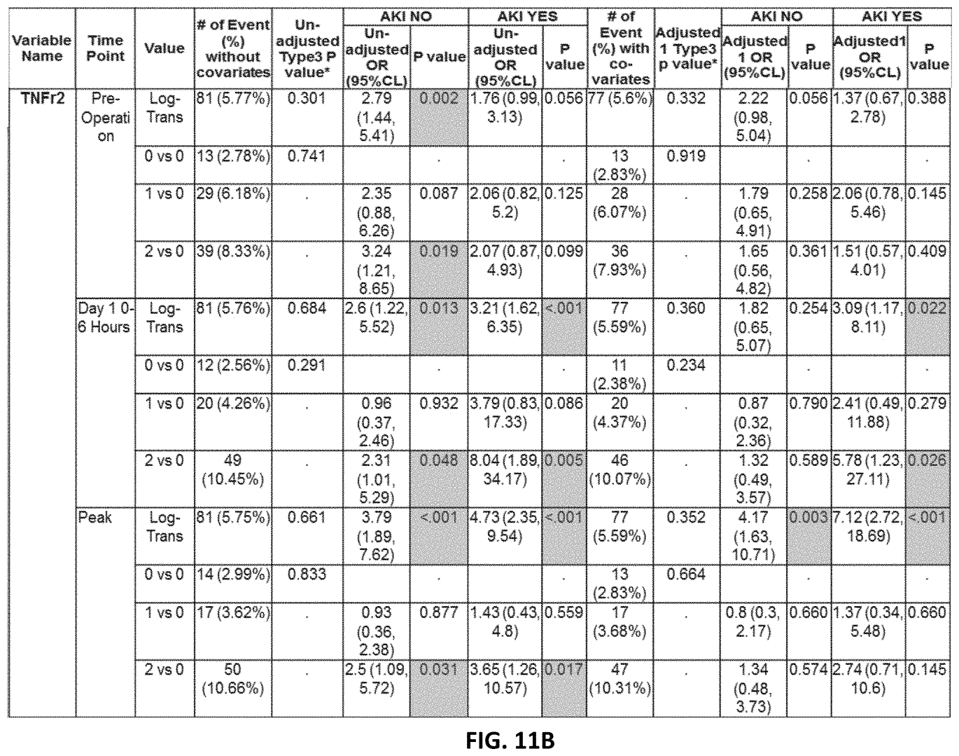

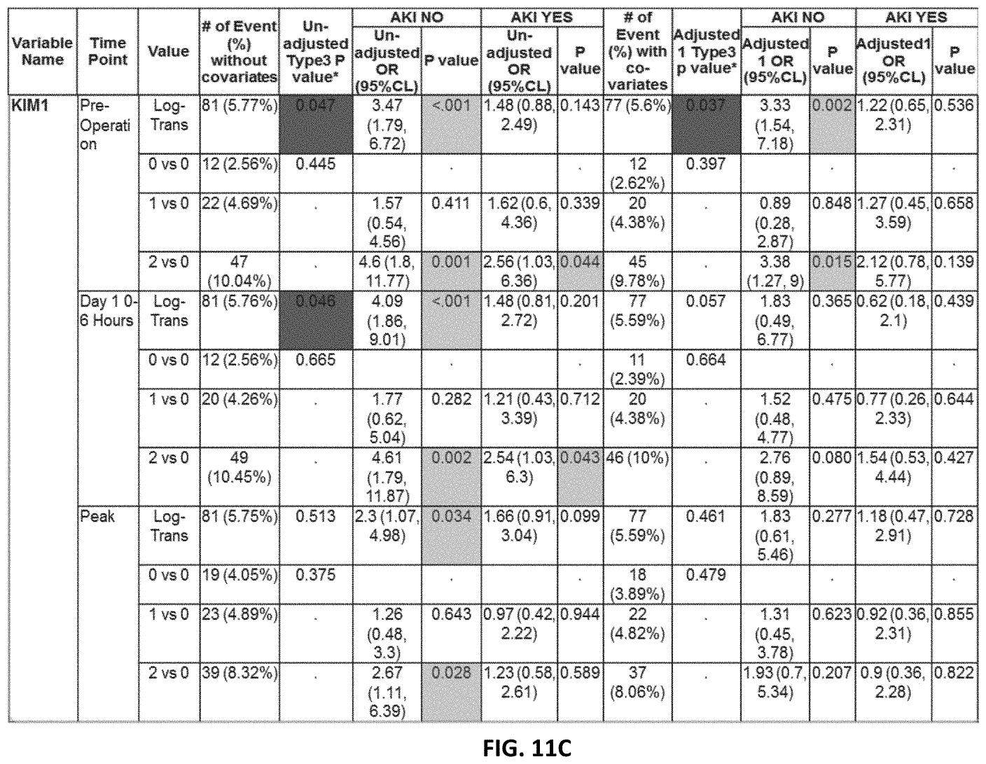

In the TRIBE-AKI cohort, plasma TNFR1, TNFR2, and KIM-1 were each independently associated with post-operative AKI and 1-year mortality in a strong, graded manner over a broad range of baseline renal function. These associations remained robust even after adjustment for several confounding variables, including baseline eGFR and CPB time (FIGS. 6-11C).

AKI is a complex, multifactorial syndrome that is a major complication of cardiac and non-cardiac surgery. Mechanistic understanding into AKI after surgery is not only the consequence of hemodynamic disturbances, but also due to inflammation. One of the key pathways that are activated in inflammatory conditions is the TNF pathway. Tumor necrosis factor is a pleotrophic cytokine that is produced predominantly by immune cells that can function in its membrane bound form or can be released as a soluble circulating 17 kDa polypeptide upon cleavage by a metalloproteinase. TNF may bind to two transmembrane receptors designated TNFR1 (pSS or CD120a) or TNFR2 (p75 or CD120b). TNFR1 is present primarily in glomeruli and endothelial cells while TNFR2 is only expressed transcriptionally in renal cells in various kidney diseases. Although TNFR2 functions as a ligand presenting receptor to TNFR1, it may have independent functions.

KIM-1 is expressed in the apical membrane of proximal tubular cells in response to injury and promotes kidney fibrosis. KIM-1 was largely studied in the urine as first a marker for AKI and subsequently as a potential biomarker for CKD. However, there is now knowledge that KIM-1 may enter the circulation because of increased transepithelial permeability or loss of epithelial cell polarity with basolateral membrane expression in acute and chronic kidney injury. In recently published studies in type 1 diabetics, plasma KIM-1 levels were independently associated with eGFR loss in patients with normal renal function at baseline and plasma KIM-1 strongly predicted ESRD risk in patients with type 1 DM and albuminuria.

There are few interventions to prevent AKI or ameliorate the consequences of AKI once it occurs. Several agents are being actively tested in clinical trials. However, due to low event rates and long follow up for event accrual, many trials are inefficient and resource-intensive. Thus, there is a great need for robust prognostic biomarkers for AKI that can allow selective enrollment of those patients with high likelihood of events that would facilitate more efficient clinical trials with higher likelihood of success. In addition, identification of these high-risk participants might be valuable for targeted enrollment in clinical trials, leading to increased events over shorter follow-up periods, ultimately culminating in shorter, efficient trial design. This is consistent with the Food and Drug Administration (FDA) guidance for enrichment strategies for clinical trials.

As demonstrated herein, TNFR1, TNFR2 and plasma KIM-1 levels were associated with higher risk of AKI and one-year mortality in patients undergoing cardiac surgery in the TRIBE-AKI cohort. The consistency of evidence in these two cohorts along with the previously published literature would suggest that the TNFRs have sufficient evidence to be considered for qualification as prognostic biomarkers for AKI in cardiac surgery. Moreover, these robust markers can be leveraged as drug development tools to facilitate targeted enrollment of higher risk patients for conducting clinical trials as well as informing better risk prediction in individual patients.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of delaying or preventing development of acute kidney injury (AKI) in a subject, the method comprising:
measuring concentrations of at least two biomarkers selected from the group consisting of tumor necrosis factor receptor-1 (TNFR1), tumor necrosis factor receptor-2 (TNFR2), and kidney injury molecule-1 (KIM-1) from a sample from the subject,
comparing the subject's biomarker concentrations with those from a reference sample, wherein, when the subject's biomarker concentrations are higher than those from the reference sample, the subject is determined to be at higher risk than average for AKI, and
administering a therapeutic treatment to the subject determined to be at higher risk than average for AKI, thereby delaying or preventing the development of AKI.

2. The method of claim 1, wherein the subject has undergone cardiac surgery.

3. The method of claim 2, wherein the subject's biomarker concentrations are measured pre-cardiac surgery.

4. The method of claim 1, wherein the therapeutic treatment is selected from the group consisting of medication, fluids, contra-statins, blood transfusions, and renal replacement therapy.

5. A method of treating AKI in a subject, the method comprising:
measuring concentrations of at least two biomarkers selected from the group consisting of TNFR1, TNFR2, and KIM-1 from a sample from the subject,
comparing the subject's biomarker concentrations with those from a reference sample, wherein, when the subject's biomarker concentrations are higher than those from the reference sample, the subject is determined to be in need of treatment for AKI, and
administering a therapeutic treatment for AKI to the subject in need thereof.

6. The method of claim 5, wherein the subject has undergone cardiac surgery.

7. The method of claim 5, wherein the subject's biomarker concentrations are measured pre-cardiac surgery.

8. The method of claim 5, wherein the therapeutic treatment is selected from the group consisting of medication, fluids, contra-statins, blood transfusions, and renal replacement therapy.

9. A method of treating a subject post-cardiac surgery, the method comprising:
measuring pre-cardiac surgery concentrations of at least two biomarkers selected from the group consisting of TNFR1, TNFR2, and KIM-1 from a sample from the subject,
measuring post-cardiac surgery concentrations of at least two biomarkers selected from the group consisting of TNFR1, TNFR2, and KIM-1 from a sample from the subject,
comparing the subject's pre-cardiac surgery biomarker concentrations with the subject's post-cardiac surgery biomarker concentrations, wherein, when the subject's pre-cardiac surgery concentrations are higher than the subject's post-cardiac surgery concentrations, the subject is determined to be at higher risk than average for mortality, and
administering a therapeutic treatment to the subject determined to be at higher risk than average for AKI.

10. The method of claim 9, wherein the therapeutic treatment is selected from the group consisting of medication, fluids, contra-statins, blood transfusions, and renal replacement therapy.

11. The method of claim 1, wherein the biomarker concentrations are measured via a bioassay.

12. The method of claim 5, wherein the biomarker concentrations are measured via a bioassay.

13. The method of claim 9, wherein the biomarker concentrations are measured via a bioassay.

14. The method of claim 1, wherein the subject is a human.

15. The method of claim 1, wherein the sample is a plasma sample.

16. The method of claim 5, wherein the sample is a plasma sample.

17. The method of claim 9, wherein the sample is a plasma sample.

18. A kit useful for assessing the level of risk for AKI in a subject, the kit comprising a bioassay capable of detecting at least one of TNFR1, TNFR2, and KIM-1, and instructional material for use thereof.
